(19) United States
(12) Patent Application Publication
(10) Pub. No.: US 2016/0220773 A1
(43) Pub. Date: Aug. 4, 2016

(54) LARYNGEAL MASK AIRWAY

(71) Applicants: Duke Chang, Hsinchu County (TW); Shen-Chih Wang, Taipei City (TW); Siou-Han Lin, Tainan City (TW); Po-Wei Hung, Hsinchu City (TW); Liang-Yi Sung, Changhua County (TW); Yi-Chang Chen, Hsinchu County (TW)

(72) Inventors: Duke Chang, Hsinchu County (TW); Shen-Chih Wang, Taipei City (TW); Siou-Han Lin, Tainan City (TW); Po-Wei Hung, Hsinchu City (TW); Liang-Yi Sung, Changhua County (TW); Yi-Chang Chen, Hsinchu County (TW)

(21) Appl. No.: 14/884,783

(22) Filed: Oct. 16, 2015

(30) Foreign Application Priority Data
Feb. 4, 2015 (TW) 104103718

(51) Int. Cl. A61M 16/04

(52) U.S. Cl. CPC A61M 16/0488; A61M 16/0402

(57) ABSTRACT

A laryngeal mask airway is provided. The laryngeal mask airway mainly includes a handle. A front end of the handle is connected to a mask body, and the handle and the mask body are formed of a flexible polymer material (e.g., polyvinyl chloride, PVC) for medical use. A guiding hole is formed at a side where the mask body and the handle are connected, and a working area is formed at an external opening of the guiding hole. With the working area, after an endotracheal intubation procedure is completed, a medical staff is able to hold the endotracheal tube with fingers throughout the whole procedure when the laryngeal mask airway is being removed, to prevent an improper movement of the endotracheal tube.

### CROSS-REFERENCE TO RELATED APPLICATION

This application claims the priority benefits of Taiwan application serial no. 104201800, filed on Feb. 4, 2015. The entirety of the above-mentioned patent application is hereby incorporated by reference herein and made a part of this specification.

### BACKGROUND OF THE INVENTION

1. Field of the Invention

The invention relates to a laryngeal mask airway (LMA), and particularly relates to an LMA in a novel structure that allows to further perform an endotracheal intubation procedure.

2. Description of Related Art

Regardless of basic emergency resuscitation or advanced cardiopulmonary resuscitation, the patency of the respiratory tract and the ventilation of the lung are crucial factors. Even though maintaining the patency of the respiratory tract and the ventilation of the lung are very difficult techniques, such techniques have the most significant impact in emergency services and are of most help to the patient once successfully carried out. In addition to endotracheal intubation, the conventional emergent respiratory tract treatment and lung ventilation procedures in emergency care include the following: i. a mouth-to-mouth or mouth-to-mask artificial respiration procedure; ii. a positive pressure ventilation procedure using a mask having an air storage bag, with assistance of an artificial oropharyngeal or nasopharyngeal respiratory tract; iii. an automatic resuscitation/ventilation procedure with a mask (with the angle of the head and the neck maintained), with assistance of an artificial oropharyngeal or nasopharyngeal respiratory tract. However, the aforementioned procedures are under different limitations to some extent. For example, i. the air may leak from between the mask and the face; ii. the air may be undesirably pumped into the stomach; iii. the respiratory tract cannot be properly protected when the substance is inhaled or aspirated into the respiratory tract or the substance in the stomach flows back; iv. when the patient suffers from a facial damage, the respiratory tract cannot be protected.

Of course, most of the limitations above may be eliminated by performing the endotracheal intubation procedure in emergency care. However, for the emergency care personnel, a timely endotracheal intubation procedure may not be necessarily carried out successfully considering the environmental limitation or the patient's condition. Besides, in practice, it is not possible to provide the most complete and detailed endotracheal intubation training to every emergency care personnel. Some countries, such as the United States, even strictly stipulate that an invasive endotracheal intubation procedure shall only be performed by a licensed medical staff. Thus, in addition to the performing the endotracheal intubation procedure in the emergency care, an emergency procedure and device for respiratory tract treatment and lung ventilation that can be performed easily without the aforementioned limitations are certainly needed.

Accordingly, a laryngeal mask airway as shown in FIG. 1 has been developed. The laryngeal mask airway includes a handle 10. One end of the handle 10 is sheathed in and connected to a mask body 11, and a mask bag 12 is disposed on a peripheral edge of the mask body 11. The mask bag 12 is connected to an inflation tube 13. The laryngeal mask airway may be used according to the following steps. First of all, a laryngeal mask airway with an appropriate size is chosen, and the air in the mask bag 12 is completely released. Then, the laryngeal mask airway is placed into the patient's mouth, with the top portion of the laryngeal mask airway facing an inner surface of the patient's upper teeth, the neck remaining flexible, and the head extending. Then, the mask bag 12 is gently pressed to be disposed at the back of the wall of the pharynx with the index finger, and then the mask bag 12 is guided to a correct position. Afterwards, the handle 10 is gently pressed downward to confirm that the mask bag 12 is fully inserted. Finally, the mask bag 12 is inflated by using the inflation tube 13, so as to form an enclosed ring pad surrounding the opening of the patient's glottis. In this way, oxygen or anesthetizing gas may be supplied to the patient's lung by using the handle 10.

Even though the conventional laryngeal mask airway helps improve the respiratory tract treatment and lung ventilation in the emergency care, further improvement is still needed and there are still issues to work on. After an emergency treatment procedure using the laryngeal mask airway is performed to the patient and the patient is diagnosed by the doctor to further require an endotracheal intubation procedure, since there is no working area at the side of the conventional laryngeal mask airway, and there is no structure for inserting an endotracheal tube designed in advance, the conventional laryngeal mask airway must be removed after the endotracheal tube is inserted to a predetermined position by using the handle 10 of the conventional laryngeal mask airway. When removing the conventional laryngeal mask airway, a stringer needs to be pressed to the endotracheal tube, so as to prevent the endotracheal tube that is inserted and positioned to a predetermined position from being carried along and moved upward during the procedure of removing the conventional laryngeal mask airway. Also, when the stringer is being pressed to the endotracheal tube, the endotracheal tube that is inserted and positioned to a predetermined position may be further pushed downward due to an excessive force exerted during the procedure of removing the conventional laryngeal mask airway. Both circumstances mentioned above may result in an undesired movement of the endotracheal tube. If the endotracheal tube is too deep, the trachea may be damaged, making only one side of the lobes of the lung receive oxygen supply while the other side of the lobes necrotic. If the endotracheal tube is not deep enough, the air supply may be insufficient. Not only the patient may be hurt, the damage may even be unrecoverable or the patient's life may be in danger. The risk of medical disputes also increases.

Furthermore, when performing the endotracheal intubation procedure, an endoscope is usually used together to avoid damages to the vocal cords, so that the endotracheal tube may be inserted to a suitable position under the condition that the vocal cords remain visible. Even though the conventional laryngeal mask airway may be used together with the endoscope, the laryngeal mask airway needs to be withdrawn by having the stringer pressed to the endotracheal tube when the conventional laryngeal mask airway is used. Thus, the stringer can only be pressed to the endotracheal tube after the endoscope placed in the endotracheal tube is withdrawn. Under such circumstance, the medical staff has to perform the intubation procedure under the condition that the vocal cords are not visible, which may easily lead to medical malpractice.

Thus, in view of the limitations of the conventional art, how to develop a novel structure with the ideal utility is certainly an issue for the researchers in relevant fields to work on.

Based on above, with years of experience in developing, designing, and manufacturing relevant products, the inventors come up with the invention with the ideal utility after careful design and evaluation.

### SUMMARY OF THE INVENTION

The invention provides a laryngeal mask airway, and aims at offering a novel emergency device for respiratory tract treatment and lung ventilation.

The present invention provides a laryngeal mask airway with a handle. There is a trench on the handle, wherein one end of the trench is located between the laryngeal mask airway and the handle, and a guiding hole is disposed at this side. The other end of the trench is located at the end of the handle having a C-shaped cross section. The endotracheal tube is conjugated to the laryngeal mask airway with two point fixations: the tip of the endotracheal tube is fixed at the guiding hole; the body of the endotracheal tube is fixed at the C-shaped end of the trench. The aforementioned fixations are temporary and easily dispatched. The laryngeal mask airway of the present invention can be used for a supraglottic airway device as a conventional LMA. If necessary, the endotracheal tube can be intubated through the guiding hole into the trachea. Then, the laryngeal mask airway and the handle can be easily removed after splitting with the endotracheal tube.

The laryngeal mask airway according to an embodiment of the invention includes a handle, and a front end of the handle is connected to a mask body. The handle and the mask body are both formed of a flexible polymer material (e.g., polyvinyl chloride (PVC)) for medical use. A guiding hole is formed at a side where the mask body and the handle are connected, and a working area is formed at an external opening of the guiding hole. A check valve with a dome shaped structure is designed in the guiding hole, which prevents air leak during ventilation while the intubation is not interfered.

The laryngeal mask airway according to an embodiment of the invention is mainly characterized as in the following. When an endotracheal intubation procedure is completed by using the guiding hole and the laryngeal mask airway is to be removed, a medical staff may hold the endotracheal tube with fingers by using the working area, and hold the handle with the other hand to slowly remove the laryngeal mask airway. When the mask body leaves the patient's larynx and touches the fingers holding the endotracheal tube, removing of the laryngeal mask airway is paused, and as the mask body is already in a distance from the patient's larynx, a position where the endotracheal tube is held is moved to a position between the mask body and the patient's larynx. Removing of the laryngeal mask airway may resume after the endotracheal tube is held again. By operating with the working area, an improper movement of the endotracheal tube caused when removing the laryngeal mask airway is prevented.

### BRIEF DESCRIPTION OF THE DRAWINGS

The accompanying drawings are included to provide a further understanding of the invention, and are incorporated in and constitute a part of this specification. The drawings illustrate embodiments of the invention and, together with the description, serve to explain the principles of the invention.

FIG. 1 is a schematic perspective view illustrating a conventional laryngeal mask airway.

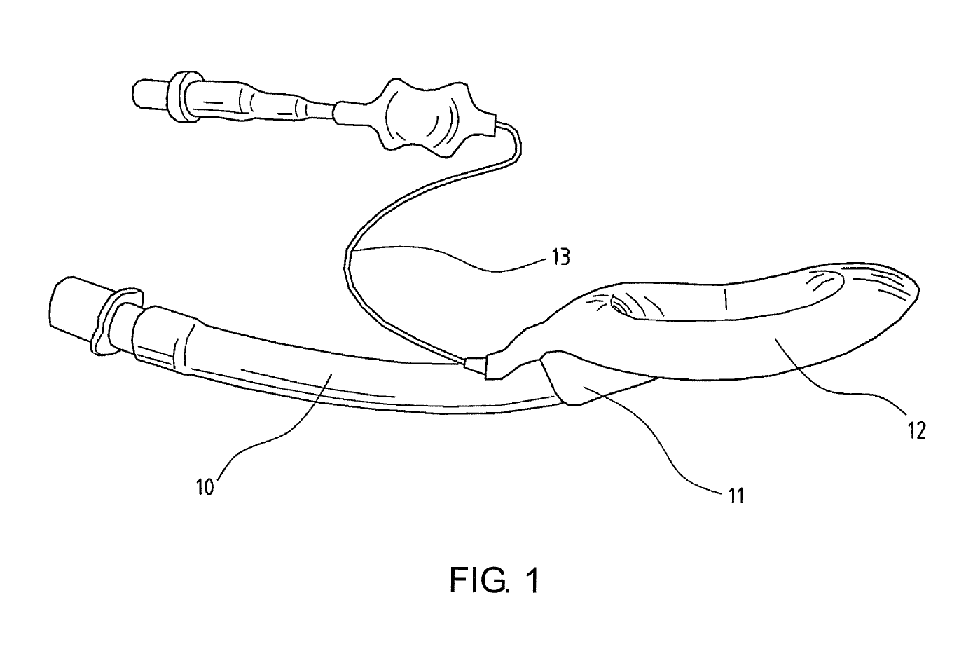

FIG. 2 is a schematic perspective view according to an embodiment of the invention.

FIG. 3 is a schematic top view according to an embodiment of the invention.

FIG. 4 is a schematic partial cross-sectional view according to an embodiment of the invention.

FIG. 5 is a schematic reference view illustrating a state of use when an embodiment of the invention is used in emergency care.

FIG. 6 is a schematic reference view illustrating a state of use when an embodiment of the invention is used in an endotracheal intubation procedure.

### DESCRIPTION OF THE EMBODIMENTS

Reference will now be made in detail to the present preferred embodiments of the invention, examples of which are illustrated in the accompanying drawings. Wherever possible, the same reference numbers are used in the drawings and the description to refer to the same or like parts.

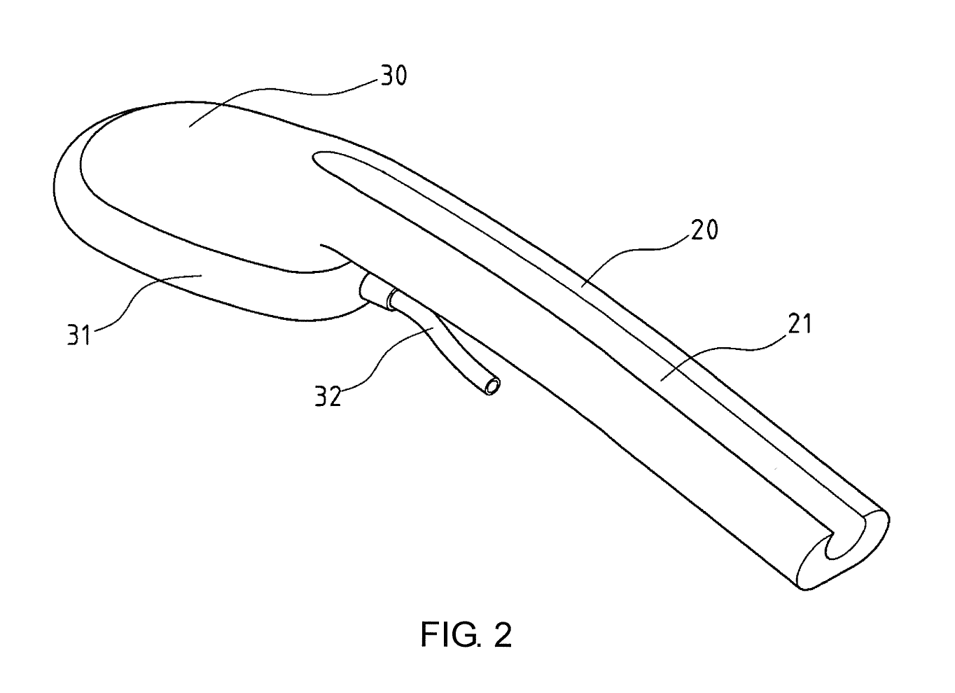

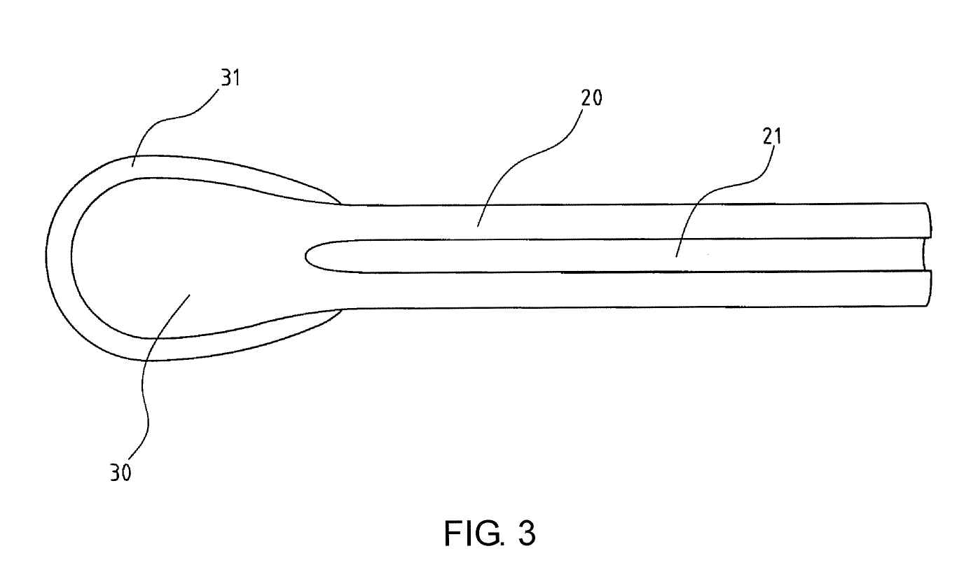

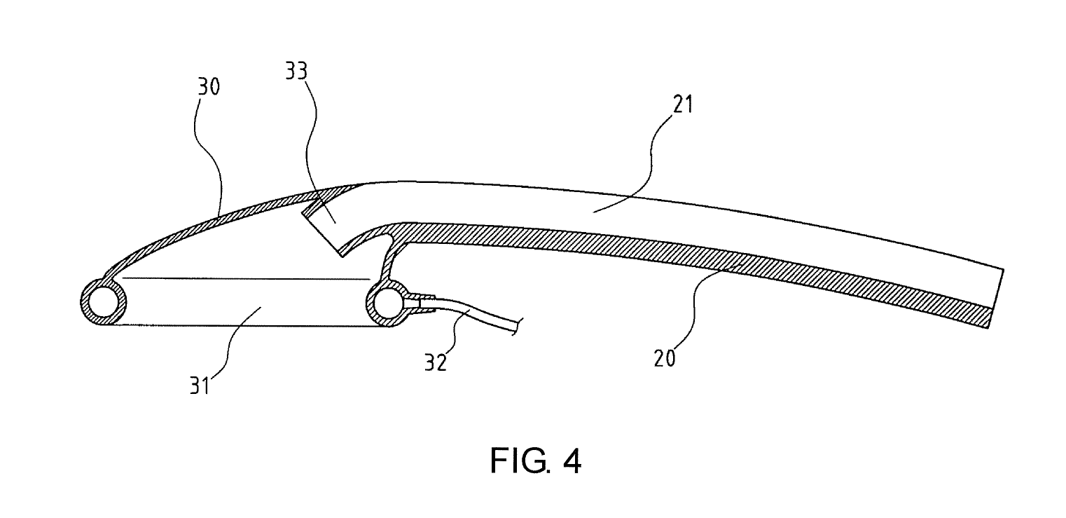

Referring to FIGS. 2, 3, and 4, FIGS. 2, 3, and 4 illustrate a larynx mask airway according to an embodiment of the invention. However, the embodiment is merely described herein for an illustrative purpose, and the invention is not limited thereto. The laryngeal mask airway includes a handle 20. A front end of the handle 20 is connected to a mask body 30. In this embodiment, the handle 20 and the mask body 30 are both formed of a flexible polymer material (e.g., polyvinyl chloride (PVC)) for medical use, and the handle 20 and the mask body 30 are integrally formed. A mask bag 31 is disposed around the periphery of the mask body 30. Besides, the bag 31 is connected to an inflation tube 32. A guiding hole 33 is disposed a side where the mask body 30 and the handle 20 are connected, and a guiding trench 21 in communication with the guiding hole 33 is formed on an exterior wall of the handle 20. A check valve (not shown) with a dome shaped structure is designed in the guiding hole, which prevents air leak during ventilation while the intubation is not interfered. The guiding trench 21 is a structure having a C-shaped cross-section and extending to a free end of the handle 20. The above embodiment describes a laryngeal mask airway structure for repetitive use. If the laryngeal mask airway is a disposable structure, the mask bag 31 and the inflation tube 32 may not be connected. In this way, the cost can be reduced without influencing the function.

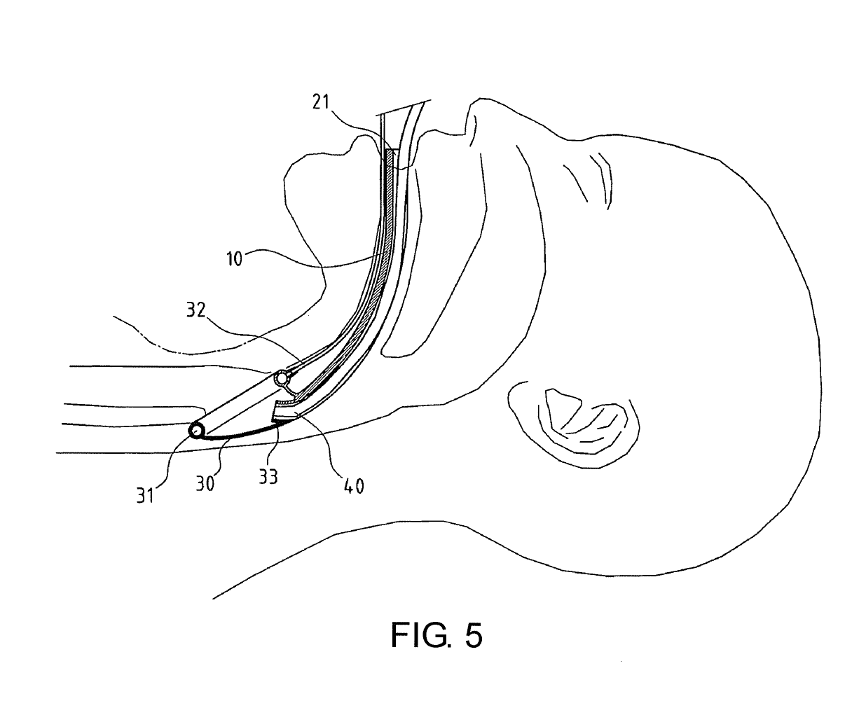

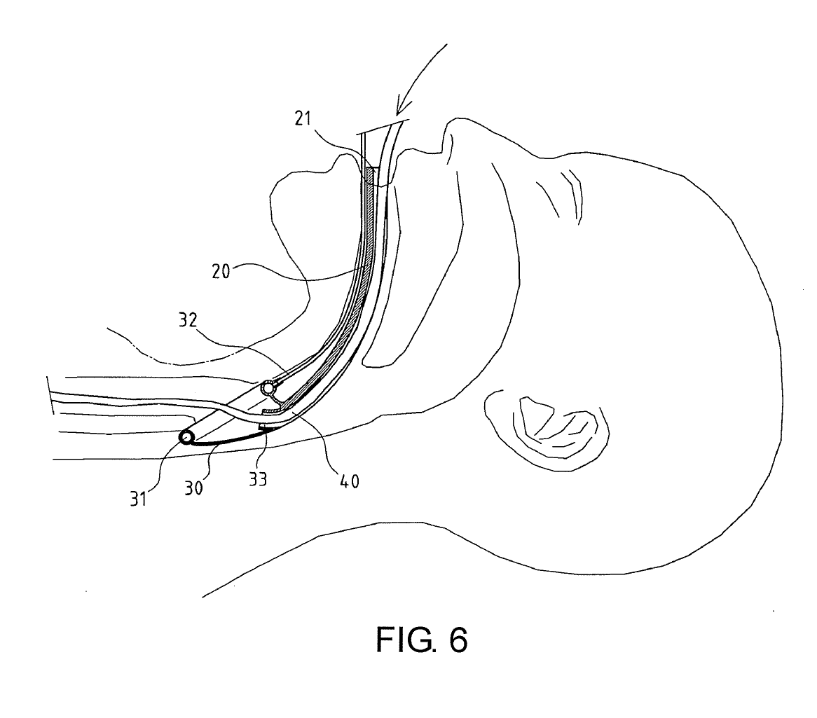

The former part of steps of using the laryngeal mask airway according to the embodiments of the invention is substantially the same as steps of using the conventional laryngeal mask airway, except that a front end of an endotracheal tube 40 is engaged into the guiding hole 33. A major difference between the steps of using the laryngeal mask airway according to the embodiments of the invention and the steps of using the conventional laryngeal mask airway lies in the endotracheal intubation procedure at the latter part of the steps. Referring FIGS. 5 and 6, when the patient using the laryngeal mask airway according to the embodiments of the invention requires an additional endotracheal intubation procedure, the intubation procedure of the endotracheal tube 40 is completed as long as the front end of the endotracheal tube 40 penetrates the mask body 30 and extends to a suitable position in the trachea. After the intubation procedure of the endotracheal tube 40 is completed, the laryngeal mask airway according to the embodiments of the invention needs to be immediately removed from the patient. To prevent the endotracheal tube 40 inserted to a predetermined position from being moved when removing the laryngeal mask airway according to the embodiments of the invention, the medical staff may gently hold the endotracheal tube 40 with his/her fingers of the right hand through the guiding trench 21, then hold the handle 20 with his/her left hand to slowly remove the laryngeal mask airway according to the embodiments of the invention. When the mask body 30 leaves the patient's larynx and touches the fingers holding the endotracheal tube 40, the removing the laryngeal mask airway according to the embodiments of the invention is paused. At this time, since the mask body 30 is already in a distance from the patient's larynx, the position where the endotracheal tube 40 is held may be moved to a position between the mask body 30 and the patient's larynx. After holding the endotracheal tube 40 again, the removing of the laryngeal mask airway according to the embodiments of the invention may resume until the laryngeal mask airway according to the embodiments of the invention is completely removed from the patient's mouth.

In view of the foregoing, when the patient using the laryngeal mask airway according to the embodiments of the invention additionally requires the endotracheal intubation procedure, the second intubation procedure is avoided, and the medical staff's fingers may gently hold the endotracheal tube 40 throughout the whole procedure when the laryngeal mask airway is being removed after the endotracheal intubation procedure is performed, so as to prevent the endotracheal tube 40 already inserted to a predetermined position from being moved during the process of pulling the laryngeal mask airway upward to remove the laryngeal mask airway. Even if the laryngeal mask airway 40 already inserted to a predetermined position is moved during the removing of the laryngeal mask airway, the medical staff is able to timely notice the movement and make correction with the fingers holding the endotracheal tube 40, while with the conventional laryngeal mask airway, the endotracheal tube can only be touched through the stringer, making the hand not sufficiently sensitive. Besides, all the procedures are performed together in a visible condition. Thus, the time required for the emergency care is reduced, and the patient's discomfort due to repetitive intubation procedures is alleviated.

Effect: With the novel structure configuration and technical feature, the laryngeal mask airway of the invention is above to provide an working area for intubation, as compared to the conventional laryngeal mask airway, such that when the laryngeal mask airway is being removed, the endotracheal tube already inserted to a predetermined position may be prevented from being moved.

It will be apparent to those skilled in the art that various modifications and variations can be made to the structure of the present invention without departing from the scope or spirit of the invention. In view of the foregoing, it is intended that the present invention cover modifications and variations of this invention provided they fall within the scope of the following claims and their equivalents.

What is claimed is:

1. A laryngeal mask airway, comprising:
a handle, wherein a front end of the handle is connected to a mask body, the handle and the mask body are formed of a flexible polymer material for medical use, wherein the flexible polymer material for medical use includes polyvinyl chloride (PVC);
a guiding hole, formed at a side where the mask body and the handle are connected; and
a guiding trench, formed on an exterior wall of the handle, and the guiding trench is in communication with the guiding hole,
wherein when an endotracheal intubation procedure is completed by using the guiding hole and the laryngeal mask airway is to be removed, a medical staff holds an endotracheal tube with fingers by using the guiding trench and holds the handle with the other hand to slowly remove the laryngeal mask airway, removing of the laryngeal mask airway is paused when the mask body leaves a patient's larynx and touches the fingers holding the endotracheal tube, and as the mask body is already in a distance from the patient's larynx, a position where the endotracheal tube is held is moved to a position between the mask body and the patient's larynx, so as to resume to remove the laryngeal mask airway after holding the endotracheal tube again, such that an improper movement of the endotracheal tube caused when removing the laryngeal mask airway is prevented.

2. The laryngeal mask airway as claimed in claim 1, wherein the guiding trench is a structure having a C-shaped cross-section and extends to a free end of the handle.

3. The laryngeal mask airway as claimed in claim 1, wherein an annular air bag is disposed on a peripheral wall of the guiding hole, and when the annular air bag is inflated, the guiding hole is closed accordingly.

4. The laryngeal mask airway as claimed in any one of claim 1, 2, or 3, wherein a mask bag is disposed around a periphery of the mask body, and the mask bag is connected to an inflation tube.
